(19) United States
(12) Patent Application Publication
(10) Pub. No.: US 2023/0003718 A1
(43) Pub. Date: Jan. 5, 2023

(54) METHODS FOR ASSESSING RATES OF DNA REPAIR

(71) Applicant: The Johns Hopkins University, Baltimore, MD (US)

(72) Inventors: Srinivasan Yegnasubramanian, Ellicott City, MD (US); Chaya Steinberg, Brooklyn, NY (US); William G. Nelson, Towson, MD (US)

(21) Appl. No.: 17/845,487

(22) Filed: Jun. 21, 2022

Related U.S. Application Data
(60) Provisional application No. 63/212,937, filed on Jun. 21, 2021.

(51) Int. Cl. G01N 33/50

(52) U.S. Cl. CPC G01N 33/5023

(57) ABSTRACT

Provided herein are methods for determining a rate of DNA double strand repair on a DNA strand in a cell that include (a) delivering a reporter gene, a gene-editing agent, and a gene-repair template into a cell, wherein the gene-editing agent generates a DNA double strand break on the DNA strand; (b) detecting a change in reporter gene expression, wherein the change in reporter gene expression indicates the presence of a DNA double strand repair event; and (c) analyzing the change in reporter gene expression, thereby determining the rate of DNA double strand repair on the DNA strand in the cell.

### CROSS-REFERENCE TO RELATED APPLICATIONS

This application claims priority to U.S. Provisional Application Ser. No. 63/212,937, filed on Jun. 21, 2021, the entire contents of which are hereby incorporated by reference.

### TECHNICAL FIELD

The present disclosure relates to the area of DNA repair pathways. In particular, it relates to DNA repair rate analysis which can assess rates of DNA double strand break repair mechanisms.

### BACKGROUND

DNA repair pathways are frequently defective in human cancers. DNA double stranded breaks (DSBs) are most often repaired by either homologous recombination (HR) or non-homologous end joining (NHEJ). Alterations in repair pathways can indicate sensitivity to therapeutic agents such as PARP inhibitors, cisplatin, and immunotherapy. Thus, functional assays to measure rates of HR and NHEJ are of significant interest. Several methods have been developed to measure rates of HR or NHEJ; however, there is a need for functional cell-based assays that can measure rates by both DNA DSB pathways simultaneously. Described herein are methods to assess rates of HR and NHEJ mediated repair of Cas9 programmed DSB simultaneously using a novel fluorescence switching reporter system, wherein the method can include a flow cytometry assay. The assay exhibits low background signal and is capable of detecting rare repair events in the 1 in 10,000 range. The utility of the assay was demonstrated by measuring the potency of inhibitors of ATM (KU-60019, KU-55933), DNA-PK (NU7441), and PARP (Olaparib) on modulating DSB repair rates in HEK293FT cells. The selective ATM inhibitor KU-60019 inhibited HR rates with IC50 of 915 nM. Interestingly, KU-60019 exposure led to a dose responsive increase in rates of NHEJ. In contrast, the less selective ATM inhibitor KU-55933, which also has activity on DNA-PK, showed inhibition of both HR and NHEJ. The selective DNA-PK inhibitor NU7441 inhibited NHEJ efficiency with an IC50 of 299 nM, and showed a dose responsive increase in HR. The PARP inhibitor Olaparib showed lower potency in modulating HR and NHEJ. The assay was then used the to assess how pharmacological and genetic inhibition of DNA methyltransferases (DNMT) impacted rates of HR and NHEJ. The DNMT inhibitor decitabine reduced HR, but increased rates of NHEJ, both in a dose responsive manner, in both HEK293FT and HCT116 cells (IC50 for HR of 187 nM and 1.4 uM respectively). Knockout of DNMT1 and DNMT3B increased NHEJ, while knockout of DNMT3B, but not DNMT1, reduced HR. These results illustrate the utility of RepairSwitch as a functional assay for measuring changes in rates of DSB repair induced by pharmacological or genetic perturbation. Furthermore, the findings illustrate the potential for one DNA repair mechanism to compensate in part for loss of another. Finally, it was shown that inhibition of DNMT can lead to reduction of HR and increase in NHEJ, providing some additional insight into recently observed synergy of DNMT inhibitors with PARP inhibitors for cancer treatment.

### SUMMARY

Provided herein are methods for determining a rate of DNA double strand repair on a DNA strand in a cell, the method comprising: (a) delivering a reporter gene, a gene-editing agent, and a gene-repair template into a cell, wherein the gene-editing agent generates a DNA double strand break on the DNA strand; (b) detecting a change in reporter gene expression, wherein the change in reporter gene expression indicates the presence of a DNA double strand repair event; and (c) analyzing the change in reporter gene expression, thereby determining the rate of DNA double strand repair on the DNA strand in the cell.

In some embodiments, the reporter gene comprises a DsRed, an EGFP, a BFP reporter gene, or any combinations thereof.

In some embodiments, the gene-editing agent comprises CRISPR/Cas9 components. In some embodiments, the gene-editing agent further comprises a guide RNA (gRNA), wherein the gRNA is targeted to an individual gene of the cell. In some embodiments, the gRNA is targeted to the EGFP reporter gene.

In some embodiments, the gene-repair template comprises an exogenous single stranded DNA oligonucleotide. In some embodiments, the exogenous single stranded DNA oligonucleotide is about 100 base pairs in length.

In some embodiments, the delivering comprises a virus-based delivery. In some embodiments, the virus-based delivery utilizes a lentivirus.

In some embodiments, the change in reporter gene expression comprises a change in fluorescence from the reporter gene. In some embodiments, the change in fluorescence comprises change from a green fluorescent protein to a blue fluorescent protein. In some embodiments, the change in fluorescence comprises loss of a green fluorescent protein.

In some embodiments, the DNA double strand repair event comprises homologous recombination (HR) or non-homologous end joining (NHEJ).

In some embodiments, the analyzing comprises flow cytometry analysis.

In some embodiments, the cell is from a HEK293FT or a HCT116 cell line.

In some embodiments, the DNA strand comprises a genetic alteration. In some embodiments, the DNA strand comprises an epigenetic alteration. In some embodiments, the epigenetic alteration comprises DNA methylation. In some embodiments, the DNA strand comprises a pharmacologic alteration. In some embodiments, the pharmacologic alteration comprises inhibition of DNA repair enzymes in the cell.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIGS. 1A-1D show exemplary schematics of cell based RepairSwitch assay to measure the balance of HR and NHEJ mediated DSB repair. (FIG. 1A) RepairSwitch assay components include: lentiviral CMV-DsRed-EF1a-EGFP vector which is transduced into cell line of interest and sorted using FACS; a LentiCRISPRv2 construct designed to target EGFP which co-expresses the Cas9 protein and the EGFP-targeting sgRNA to induce a DSB (PAM sequence in purple); and a BFP homologous recombination template protected at both ends by two consecutive phosphorothioate bonds (represented by asterisks). Sequence alignment of EGFP and BFP show that two base substitutions (orange) correspond to the two changes in amino acid sequence responsible for the shift in fluorescence from EGFP to BFP. (FIG. 1B) Rates of HR and NHEJ are determined by assessment of DsRed+ cells for EGFP and BFP fluorescence using flow cytometry. (FIG. 1C) Cells were gated on High DsRed+ expression. For each sample, 10,000-30,000 High DsRed+ cells were counted and statistical analysis was done in FlowJo and GraphPad. Representative Flow cytometry plots depict assay results and gating strategy. (FIG. 1D) This assay was performed in HEK239FT and HCT116 cells. In each experiment, the HR template and gRNA were each administered alone and served as assay controls.

FIGS. 2A-2B show impact of genetic modulation of repair factors on rates of repair. CWR 22Rv1 WT and CWR 22Rv1 ATM KO cells were transduced with the assay vectors and sorted prior to assay performance. Rates of HR (FIG. 2A) and NHEJ (FIG. 2B) were compared in these cell lines and statistical analysis was performed using an unpaired t-test.

FIGS. 3A-3D show impact of ATM inhibition on rates of repair. Rates of HR (FIG. 3A) and NHEJ (FIG. 3B) in response to inhibition of ATM using a potent and selective inhibitor, KU-60019, with an IC50 of 915 nM. Rates of HR (FIG. 3C) and NHEJ (FIG. 3D) in response to inhibition of ATM using a less potent and less selective inhibitor with off targeting of DNA-PK at higher doses, KU-55933, with an IC50 of 2.6 uM.

FIGS. 4A-4B show impact of DNA-PK inhibition on rates of repair. Rates of HR (FIG. 4A) and NHEJ (FIG. 4B) in response to inhibition of DNA-PK with a potent and selective inhibitor, NU-7441, with an IC50 of 299 nM.

FIGS. 5A-5B show impact of PARP inhibition on rates of repair. Rates of HR (FIG. 5A) and NHEJ (FIG. 5B) in response to inhibition of PARP with a potent and selective inhibitor, Olaparib, with an IC50 of 739 nM.

FIGS. 6A-6D show impact of donor template DNA methylation context on rates of repair. Rates of HR and NHEJ are shown in HEK293FT cells (FIGS. 6A-6B) and HCT116 WT (FIGS. 6C-6D) cells, respectively, with both methylated and unmethylated templates along with all other assay components. Statistical analysis was performed using unpaired t-tests.

FIGS. 7A-7D show impact of Decitabine on rates of repair. Rates of HR and NHEJ in response to Decitabine treatment in both HEK293FT (FIGS. 7A-7B) and HCT116 cells (FIGS. 7C-7D).

FIGS. 8A-8B show impact of genetic manipulation of DNA methylation pathway on rates of repair. This assay was performed in HCT116 WT, HCT116 DNMT1 KO, and HCT116 DNMT3B KO cell lines and rates of HR (FIG. 8A) and NHEJ (FIG. 8B) are shown for all assay components. Rates of HR and NHEJ were compared between these cell lines and statistical analysis was performed using unpaired t-tests.

FIGS. 9A-9D show graphs from Incucyte Proliferation Assay. Growth curves for (FIG. 9A) KU-60019 ATM inhibitor (FIG. 9B) KU-55933 ATM inhibitor (FIG. 9C) NU-7441 DNA-PK inhibitor and (FIG. 9D) Olaparib PARP inhibitor, to ascertain toxicity and growth inhibition.

FIGS. 10A-10B show graphs from Incucyte Proliferation Assay. Incucyte was performed on both (FIG. 10A) HEK293FT cells and (FIG. 10B) HCT116 cells to ascertain the toxicity and growth inhibition post Decitabine treatment.

### DETAILED DESCRIPTION

Double strand breaks, which are particularly genotoxic, can be repaired by multiple mechanisms, including homologous recombination, non-homologous end joining, single strand annealing, and microhomology end joining. However, the two mechanisms utilized most often to repair DNA double stranded breaks (DSBs) are homologous recombination (HR) and non-homologous end joining and other end joining pathways (NHEJ). Described herein are functional assays to measure rates of homologous recombination (HR) and non-homologous end joining (NHEJ) pathways. The assays can be referred to as a “RepairSwitch assay”, which is a flow cytometry assay to assess rates of HR and NHEJ mediated repair of Cas9 programmed DSB simultaneously using a novel fluorescence switching reporter system.

Provided herein are methods for determining a rate of DNA double strand repair on a DNA strand in a cell, the method comprising: (a) delivering a reporter gene, a gene-editing agent, and a gene-repair template into a cell, wherein the gene-editing agent generates a DNA double strand break on the DNA strand; (b) detecting a change in reporter gene expression, wherein the change in reporter gene expression indicates the presence of a DNA double strand repair event; and (c) analyzing the change in reporter gene expression, thereby determining the rate of DNA double strand repair on the DNA strand in the cell.

Various non-limiting aspects of these methods are described herein, and can be used in any combination without limitation. Additional aspects of various components of methods for identifying the presence or absence of a mutation and methylation are known in the art.

It must be noted that, as used in the specification and the appended claims, the singular forms “a,” “an” and “the” include plural referents unless the context clearly dictates otherwise.

As used herein, a “cell” can refer to either a prokaryotic or eukaryotic cell, optionally obtained from a subject or a commercially available source.

As used herein, “delivering”, “gene delivery”, “gene transfer”, “transducing” can refer to the introduction of an exogenous polynucleotide into a host cell, irrespective of the method used for the introduction. Such methods include a variety of well-known techniques such as vector-mediated gene transfer (e.g., viral infection/transfection, or various other protein-based or lipid-based gene delivery complexes) as well as techniques facilitating the delivery of “naked” polynucleotides (e.g., electroporation, “gene gun” delivery and various other techniques used for the introduction of polynucleotides). The introduced polynucleotide may be stably or transiently maintained in the host cell. Stable maintenance typically requires that the introduced polynucleotide either contains an origin of replication compatible with the host cell or integrates into a replicon of the host cell such as an extrachromosomal replicon (e.g., a plasmid) or a nuclear or mitochondrial chromosome.

In some embodiments, a polynucleotide can be inserted into a host cell by a gene delivery molecule. Examples of gene delivery molecules can include, but are not limited to, liposomes, micelles biocompatible polymers, including natural polymers and synthetic polymers; lipoproteins; polypeptides; polysaccharides; lipopolysaccharides; artificial viral envelopes; metal particles; and bacteria, or viruses, such as baculovirus, adenovirus and retrovirus, bacteriophage, cosmid, plasmid, fungal vectors and other recombination vehicles typically used in the art which have been described for expression in a variety of eukaryotic and prokaryotic hosts, and may be used for gene therapy as well as for simple protein expression.

As used herein, the term “exogenous” refers to any material introduced from or originating from outside a cell, a tissue or an organism that is not produced by or does not originate from the same cell, tissue, or organism in which it is being introduced.

As used herein, the term “expression” refers to the process by which polynucleotides are transcribed into mRNA and/or the process by which the transcribed mRNA is subsequently translated into peptides, polypeptides, or proteins. In some embodiments, if the polynucleotide is derived from genomic DNA, expression may include splicing of the mRNA in a eukaryotic cell. The expression level of a gene may be determined by measuring the amount of mRNA or protein in a cell or tissue sample; further, the expression level of multiple genes can be determined to establish an expression profile for a particular sample.

As used herein, “nucleic acid” is used to include any compound and/or substance that comprise a polymer of nucleotides. In some embodiments, a polymer of nucleotides are referred to as polynucleotides. Exemplary nucleic acids or polynucleotides can include, but are not limited to, ribonucleic acids (RNAs), deoxyribonucleic acids (DNAs), threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization) or hybrids thereof. Naturally-occurring nucleic acids generally have a deoxyribose sugar (e.g., found in deoxyribonucleic acid (DNA)) or a ribose sugar (e.g., found in ribonucleic acid (RNA)).

A nucleic acid can contain nucleotides having any of a variety of analogs of these sugar moieties that are known in the art. A deoxyribonucleic acid (DNA) can have one or more bases selected from the group consisting of adenine (A), thymine (T), cytosine (C), or guanine (G), and a ribonucleic acid (RNA) can have one or more bases selected from the group consisting of uracil (U), adenine (A), cytosine (C), or guanine (G).

In some embodiments, the term “nucleic acid” refers to a deoxyribonucleic acid (DNA) or ribonucleic acid (RNA), or a combination thereof, in either a single- or double-stranded form. Unless specifically limited, the term encompasses nucleic acids containing known analogues of natural nucleotides that have similar binding properties as the reference nucleotides. Unless otherwise indicated, a particular nucleic acid sequence also implicitly encompasses complementary sequences as well as the sequence explicitly indicated. In some embodiments of any of the isolated nucleic acids described herein, the isolated nucleic acid is DNA. In some embodiments of any of the isolated nucleic acids described herein, the isolated nucleic acid is RNA.

Modifications can be introduced into a nucleotide sequence by standard techniques known in the art, such as site-directed mutagenesis and polymerase chain reaction (PCR)-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., arginine, lysine and histidine), acidic side chains (e.g., aspartic acid and glutamic acid), uncharged polar side chains (e.g., asparagine, cysteine, glutamine, glycine, serine, threonine, tyrosine, and tryptophan), nonpolar side chains (e.g., alanine, isoleucine, leucine, methionine, phenylalanine, proline, and valine), beta-branched side chains (e.g., isoleucine, threonine, and valine), and aromatic side chains (e.g., histidine, phenylalanine, tryptophan, and tyrosine), and aromatic side chains (e.g., histidine, phenylalanine, tryptophan, and tyrosine).

As used herein, the term “nucleotides” and “nt” are used interchangeably herein to generally refer to biological molecules that comprise nucleic acids. Nucleotides can have moieties that contain the known purine and pyrimidine bases. Nucleotides may have other heterocyclic bases that have been modified. Such modifications include, e.g., methylated purines or pyrimidines, acylated purines or pyrimidines, alkylated riboses, or other heterocycles. The terms “polynucleotides,” “nucleic acid,” and “oligonucleotides” can be used interchangeably. They can refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise non-naturally occurring sequences. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

As used herein, a “primer” is generally a polynucleotide molecule comprising a nucleotide sequence (e.g., an oligonucleotide), generally with a free 3′-OH group, that hybridizes with a template sequence (such as a target polynucleotide, or a primer extension product) and is capable of promoting polymerization of a polynucleotide complementary to the template. In some embodiments, a primer is a biotinylated primer.

### DNA Double Strand Break

As used herein, “DNA double strand break” can refer to an event in which both strands in the double helix are severed. DNA double strand breaks can be hazardous to the cell because they can lead to genome rearrangements. In some embodiments, double strand breaks, which can be particularly genotoxic, can be repaired by multiple mechanisms, including homologous recombination, non-homologous end joining, single strand annealing, and microhomology end joining. However, the two mechanisms utilized most often to repair DNA double stranded breaks (DSBs) are homologous recombination (HR) and non-homologous end joining and other end joining pathways (NHEJ). In some embodiments, HR can be a conservative process that uses a homologous template (e.g., the sister chromatid in S or G2 phases of the cell cycle, the homologous chromosome), to repair the damaged section of DNA. In some embodiments, end joining pathways, including NHEJ, can be error prone and often mutagenic. In some embodiments, NHEJ involves ligation of ends of the broken DNA, often adding or deleting nucleotides prior to ligation, which results in deletions and frameshifts.

In some embodiments, a DNA double strand break can be repaired by a DNA double strand repair event. In some embodiments, the DNA double strand repair event can be homologous recombination, non-homologous end joining, single strand annealing, or microhomology end joining. In some embodiments, the DNA double strand repair event can be homologous recombination (HR). In some embodiments, the DNA double strand repair event can be non-homologous end joining (NHEJ).

### Method for Determining a Rate of DNA Double Strand Repair

Provided herein are methods for determining a rate of DNA double strand repair on a DNA strand in a cell that include (a) delivering a reporter gene, a gene-editing agent, and a gene-repair template into a cell, wherein the gene-editing agent generates a DNA double strand break on the DNA strand; (b) detecting a change in reporter gene expression, wherein the change in reporter gene expression indicates the presence of a DNA double strand repair event; and (c) analyzing the change in reporter gene expression, thereby determining the rate of DNA double strand repair on the DNA strand in the cell.

As used herein, a “reporter gene” can refer to an exogenous gene that can be used to detect and measure gene expression, wherein when a reporter gene is introduced into a target cell (e.g., brain cell, cancer cell) it produces a protein receptor or enzyme that binds or transports an imaging probe. In some embodiments, a reporter gene can be used to track the physical location of a segment of DNA or to monitor gene expression. In some embodiments, examples of a reporter gene can include, but are not limited to, lacZ gene, cat gene, gfp gene, rfp gene, or luc gene. In some embodiments, a reporter gene can include a DsRed, an EGFP, a BFP reporter gene, or any combinations thereof.

In some embodiments, a change in reporter gene expression can indicate the presence of a DNA double strand repair event. In some embodiments, the change in reporter gene expression comprises a change in fluorescence from the reporter gene. In some embodiments, the change in fluorescence comprises change from a green fluorescent protein to a blue fluorescent protein. In some embodiments, the change in fluorescence comprises loss of a green fluorescent protein.

As used herein, a “gene-editing agent” can refer to an agent that can target and bind to a specific sequence in DNA. In some embodiments, a gene-editing agent comprises CRISPR/Cas9 components. As used herein, the term “CRISPR” refers to a technique of sequence specific genetic manipulation relying on the clustered regularly interspaced short palindromic repeats pathway, which unlike RNA interference regulates gene expression at a transcriptional level. As used herein, a “Cas effector” or “CRISPR-associated protein” can refer to an enzyme or protein that uses CRISPR sequences as a guide to recognize and cleave specific nucleic acid strands that are complementary to the CRISPR sequence. A gene-editing Cas effector can associate with a CRISPR RNA sequence to bind to, and alter DNA or RNA target sequences. In some embodiments, the gene-editing agent comprises a gene-editing Cas effector. In some embodiments, the gene-editing Cas effector comprises a Cas9 protein, a Cas13b protein, or a Cas13d protein. In some embodiments, a gene-editing Cas effector can be a Cas9 endonuclease that makes a double-stranded break in a target DNA sequence. In some embodiments, a gene-editing Cas effector can be a Cas12a nuclease that also makes a double-stranded break in a target DNA sequence. In some embodiments, a gene-editing Cas effector can be a Cas13 nuclease which targets RNA. In some embodiments, a gene-editing Cas effector comprises a Cas9 protein, a Cas13b protein, or a Cas13d protein. In some embodiments, the gene-editing Cas effector comprises a nuclease dead Cas9 (dCas9) protein. In some embodiments, the gene-editing Cas effector comprises a Cas13b protein. In some embodiments, the gene-editing Cas effector comprises a Cas13d protein.

In some embodiments, the gene-editing agent further comprises a guide RNA (gRNA), wherein the gRNA is targeted to an individual gene of a cell. The term “guide RNA” or “gRNA” is a specific type of gRNA that combines tracrRNA (transactivating RNA), which binds to Cas9 to activate the complex to create the necessary strand breaks, and crRNA (CRISPR RNA), comprising complimentary nucleotides to the tracrRNA, into a single RNA construct. Exemplary methods of employing the CRISPR technique are described in WO 2017/091630, which is incorporated by reference in its entirety.

In some embodiments, the guide RNA can recognize a target RNA, for example, by hybridizing to the target RNA. In some embodiments, the guide RNA comprises a sequence that is complementary to the target RNA. In some embodiments, the gRNA can include one or more modified nucleotides. In some embodiments, the gRNA has a length that is about 10 nt (e.g., about 20 nt, about 30 nt, about 40 nt, about 50 nt, about 60 nt, about 70 nt, about 80 nt, about 90 nt, about 100 nt, about 120 nt, about 140 nt, about 160 nt, about 180 nt, about 200 nt, about 300 nt, about 400 nt, about 500 nt, about 600 nt, about 700 nt, about 800 nt, about 900 nt, about 1000 nt, or about 2000 nt).

In some embodiments, the gene-editing agent comprises a guide RNA (gRNA), wherein the gRNA is targeted to an individual gene of the cell. In some embodiments, the gRNA is targeted to the EGFP reporter gene. In some embodiments, the gRNA can be driven by a promoter. In some embodiments, the promoter can be a U6 polymerase III promoter.

As used herein, a “gene-repair template” can refer to a homologous DNA template to serve as a primer for DNA repair synthesis. In some embodiments, a gene-repair template can come from within the cell during late S phase or G2 phase of the cell cycle, when sister chromatids are available prior to the completion of mitosis. Additionally, in some embodiments, exogenous repair templates can be delivered into a cell, most often in the form of a synthetic, single-strand DNA donor oligo or donor plasmid, to generate a precise change in the genome. In some embodiments, the gene-repair template can be a plasmid donor repair template, wherein the repair template is delivered into a cell to insert or change large sequences (e.g., knock-in a fluorescent reporter or replace an entire gene) of DNA in the endogenous genomic target region. In some embodiments, the gene-repair template comprises an exogenous single stranded DNA oligonucleotide. In some embodiments, the exogenous single stranded DNA oligonucleotide is about 100 base pairs in length. In some embodiments, the exogenous single stranded DNA oligonucleotide is about 20 to about 200 (e.g., about 40 to about 200, about 60 to about 200, about 80 to about 200, about 100 to about 200, about 120 to about 200, about 140 to about 200, about 160 to about 200, about 180 to about 200, about 20 to about 180, about 40 to about 180, about 60 to about 180, about 80 to about 180, about 100 to about 180, about 120 to about 180, about 140 to about 180, about 160 to about 180, about 20 to about 160, about 40 to about 160, about 60 to about 160, about 80 to about 160, about 100 to about 160, about 120 to about 160, about 140 to about 160, about 20 to about 140, about 40 to about 140, about 60 to about 140, about 80 to about 140, about 100 to about 140, about 120 to about 140, about 20 to about 120, about 40 to about 120, about 60 to about 120, about 80 to about 120, about 100 to about 120, about 20 to about 100, about 40 to about 100, about 60 to about 100, about 80 to about 100, about 20 to about 80, about 40 to about 80, about 60 to about 80, about 20 to about 60, about 40 to about 60, or about 20 to about 40) base pairs in length.

In some embodiments, the methods described herein comprise delivering a reporter gene, a gene-editing agent, and a gene-repair template into a cell. As used herein, “delivering” refers to the introduction of an exogenous polynucleotide into a cell. Such methods include a variety of well-known techniques such as vector-mediated gene transfer (e.g., viral infection/transfection, or various other protein-based or lipid-based gene delivery complexes). In some embodiments, the delivering comprises a virus-based delivery. In some embodiments, the virus-based delivery utilizes a lentivirus.

In some embodiments, the methods described herein comprise analyzing the change in reporter gene expression, thereby determining the rate of DNA double strand repair on the DNA strand in the cell. In some embodiments, the analyzing comprises flow cytometry analysis. In some embodiments, determining a level of gene expression can include chemiluminescence or fluorescence techniques. In some embodiments, determining a level of gene expression can include immunological-based methods (e.g., quantitative enzyme-linked immunosorbent assays (ELISA), Western blotting, or dot blotting) wherein antibodies are used to react specifically with entire proteins or specific epitopes of a protein. In some embodiments, determining a level of gene expression can include immunoprecipitation of the protein.

In some embodiments, the cell can be from eukaryotic cells. In some embodiments, the cell can be from cancer cells. In some embodiments, the cell is from a HEK293FT or a HCT116 cell line. In some embodiments, the cell can have functional loss of DNA double strand repair. In some embodiments, the cell can have functional loss of DNA double strand repair due to genetic alterations to the DNA. In some embodiments, the genetic alterations can include mutations or copy number alterations. In some embodiments, the cell can have functional loss of DNA double strand repair due to epigenetic alterations. In some embodiments, the epigenetic alterations can include DNA methylation, changes in chromatin structure, or histone modification. In some embodiments, the cell can have functional loss of DNA double strand repair due to pharmacologic alteration. In some embodiments, the pharmacologic alteration can include drug exposure or pharmacological inhibition of gene expression.

In some embodiments, the DNA strand in the cell can include a genetic alteration. In some embodiments, the genetic alteration can include a mutation. In some embodiments, the DNA strand includes an epigenetic alteration. In some embodiments, the epigenetic alteration comprises DNA methylation. In some embodiments, the DNA strand includes a pharmacologic alteration. In some embodiments, the pharmacologic alteration comprises inhibition of DNA repair enzymes in the cell due to drug exposure.

### EXAMPLES

The disclosure is further described in the following examples, which do not limit the scope of the disclosure described in the claims.

### Plasmid Construction

Vectors for this reporter assay were constructed using a lentiviral construct containing CMV-DsRed and UBC-EGFP on a pHAGE backbone purchased from Addgene (plasmid #24526). This lentiviral construct then underwent site directed mutagenesis using the Agilent QuikChange II kit, introducing an AgeI cut site at the c-terminus of the UBC promoter (3897-3901) via 3 base changes. The plasmid was then restriction digested by AgeI and BamHI to remove the UBC promoter via gel extraction. DNA encoding the EF1a promoter was PCR amplified using NEB Phusion HF DNA Polymerase Kit, attaching AgeI and BamHI restriction sites to the 3′ and 5′ ends, respectively, and was subsequently digested. This amplicon was then cloned into the pHAGE backbone using cohesive ligation via a Quick Ligase Kit.

Plasmids expressing DsRed, EGFP, and BFP individually were constructed as compensation control for flow cytometry using this backbone. The DsRed only plasmid was constructed via restriction digestion by BamHI and ClaI and subsequent gel extraction of the reporter assay construct, to remove the EF1a-UBC region. A short, ˜30 bp oligo, purchased from IDT with BamHI and ClaI restriction sites on the 5′ and 3′ ends, respectively, was then digested and used to cohesively ligate the ends via Quick Ligase Kit. To create the EGFP only construct, the reporter assay construct was restriction digested using SpeI and BamHI followed by gel extraction to remove CMV-DsRed. The resulting backbone was treated with Mung Bean Nuclease and a Quick Ligase Kit was used to circularize the plasmid via blunt end ligation. This plasmid then underwent site directed mutagenesis using the Agilent QuikChange II Kit to create the BFP only plasmid (within EGFP: C197G_T199C).

### Viral Packaging

Lentiviral packaging vectors pMDLg/pRRE (5 ug), pRSV-Rev (2.5 ug), and pMD2.G (2.5 ug) were used to package viruses of all assay vectors (10 ug of vector). FuGENE HD was used to transfect packaging vectors and assay constructs into HEK293FT cells for viral production in a ratio of 3:1 FuGENE to DNA. Media was replaced 4-16 hours post transfection. Virus was collected 48-hours post transfection and then 12-hours later, combining both batches. Virus was then spun down, aliquoted, and stored at −80 C.

### Transduction, MOI, and FACS

Spinfection protocol was used to transduce cell lines, adding 8 ug/ml polybrene to the growth medium along with virus followed by centrifugal spin for 2 hours before being placed in the incubator. Media was replaced 24-hours post transduction and flow cytometry and FACS was performed after 72-96 hours. MOI of 0.3 was assessed using viral dose curve and flow cytometry. FACS was used to sort positive cell populations.

### Gene Targeting

A Cas9/gRNA vector targeting EGFP was constructed using the LentiCRISPRV2 backbone and their established protocol for gRNA production. The EGFP-targeting gRNA sequence was designed using ChopChop.

### HR Repair Template

ssDNA oligo containing BFP homologous template to GFP was purchased from IDT and resuspended to a 1 ug/ul concentration. This repair template is ˜100 bp in length, protected at 5′ and 3′ ends with two consecutive phosphorothioate bonds. Methylated versions of this template were also purchased containing methyl groups at all available CpGs, seven sites in total. Methylated versions of the aforementioned ssDNA oligo template were also purchased from IDT and resuspended to a 1 ug/ul concentration. These methylated oligos contain methyl groups at all available CpGs, seven sites in total. This repair template is also ˜100 bp in length and protected at 5′ and 3′ ends with two consecutive phosphorothioate bonds.

### Cell Culture

HEK293FT cells were obtained from ThermoFisher Scientific (R70007) and subsequently cultured in DMEM, a high glucose medium supplemented with 10% FBS. HCT116 DNMT1 KO and DNMT3B KO cell lines were cultured in McCoy's growth medium supplemented with 10% FBS. CWR22RV1 cells and CWR22RV1 ATM−/− cells were cultured in RPMI-1640 growth medium supplemented with 10% FBS.

Cell Line Transfections with CRISPR/Cas9 Reagents and HR Repair Template

All cell lines were plated in 12-well plates at 2.5E5 cells/well in their respective growth mediums. The growth medium was then replaced after 24-48 hours and the cells were transfected with lug of CRISPR/Cas9 gRNA construct, lug of HR repair template, or both gRNA and HR repair template (2 ug total) in combination using FuGENE HD (Promega) at a FuGENE to DNA ratio of 3:1. The DNA was mixed with Opti-MEM (50 ul Opti-MEM/ug DNA) and FuGENE was added for an incubation period of 15 minutes before being added dropwise to the well. If drug is being utilized, cells are dosed immediately prior to transfection. Fresh growth medium was replaced every 48-72 hours. Cells were kept in culture for 1 week before assessment of fluorescence via flow cytometry.

### Pharmacological Inhibitors

All compounds used can be found listed in Table 1, and all were purchased from Selleck. All drugs were dissolved in DMSO as per Selleck instructions. For cell-based work drugs were diluted to a final concentration of 0.001% DMSO. All drugs were given in a dose range of 1 nm to 10 uM, unless toxicity was shown in Incucyte proliferation assays (S1, S2).

TABLE 1

| | | | IC50 | | | |
|---|---|---|---|---|---|---|
| | | Target | Cell-free | | Off Target | |
| | Target | Potency | Assay | Off Target | IC50 | Structure |
| KU-60019 | ATM | ++++ | 6.3 nM | — | — | 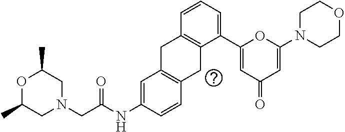 [SMILES: C[C@@H]1CN(CC(=O)Nc2ccc3c(c2)Cc2cccc(-c4cc(=O)cc(N5CCOCC5)o4)c2C3)C[C@H](C)O1] |
| KU-55933 | ATM | +++ | 12.9 nM | DNA-PK | 2.5 uM | 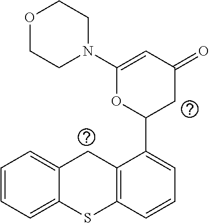 [SMILES: O=C1C=C(N2CCOCC2)OC(c2cccc3c2Cc2ccccc2S3)C1] |
| NU-7441 | DNA-PK | ++ | 14 nM | P13K | 5 uM | 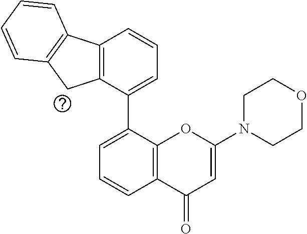 [SMILES: O=c1cc(N2CCOCC2)oc2c(-c3cccc4c3Cc3ccccc3-4)cccc12] |
| Olaparib | PARP1/2 | ++/++++ | 5 nM/1 nM | — | — | 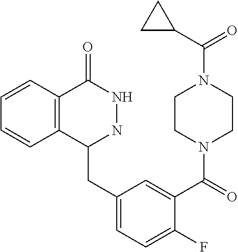 [SMILES: O=C1NNC(Cc2ccc(F)c(C(=O)N3CCN(C(=O)C4CC4)CC3)c2)c2ccccc21] |
| Decitabine | DNMT | ++++ | N/A | — | — | 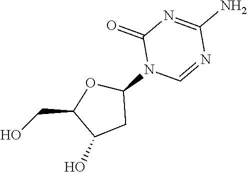 [SMILES: Nc1ncn([C@H]2C[C@H](O)[C@@H](CO)O2)c(=O)n1] |

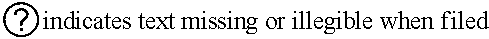

### Flow Cytometry Analysis

Medium was removed from 12-well plates and cells were detached from plate using TrypLE Express. FBS containing medium was used to neutralize TrypLE Express and cells were collected and spun down at 1000 RPM for 5 min. Cell pellets were then washed with PBS twice before resuspension in 500 ul PBS and put on ice. Prior to flow of each sample, cells were resuspended and put through a cell-strainer cap on the flow tube. Cells were gated to ensure single cell population as well as high DsRed expression. For each sample, 10,000-30,000 high DsRed expressing cells were counted depending on sample concentration, and assessed for both EGFP and BFP expression. FlowJo was utilized to analyze all flow cytometry results and to produce statistics on each population.

### Visualization and Statistical Analysis

Graphpad Prism was utilized to visualize statistical results from FlowJo. Additionally, it was utilized to provide statistical analyses of p value using unpaired t-tests.

### Example 1—Overview of RepairSwitch Assay Principles

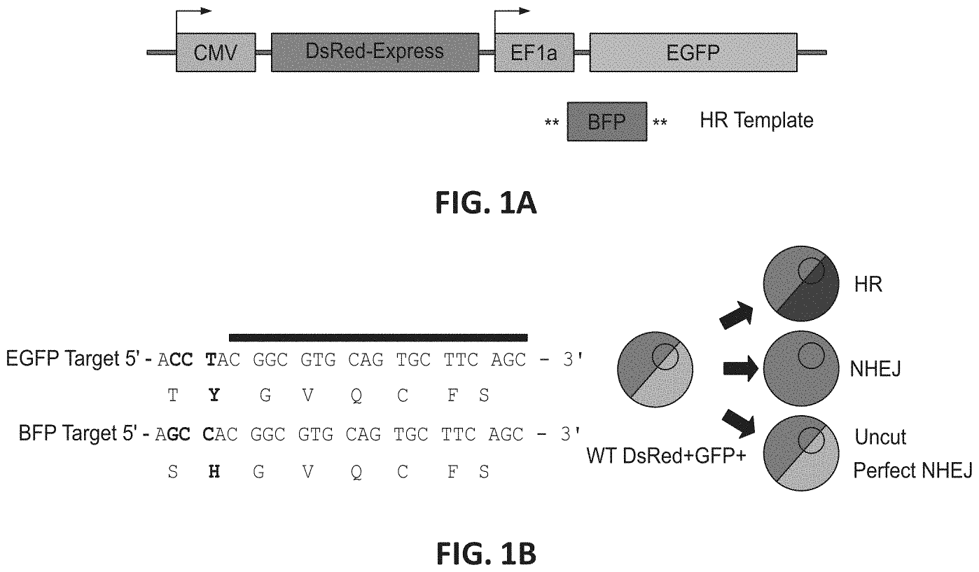

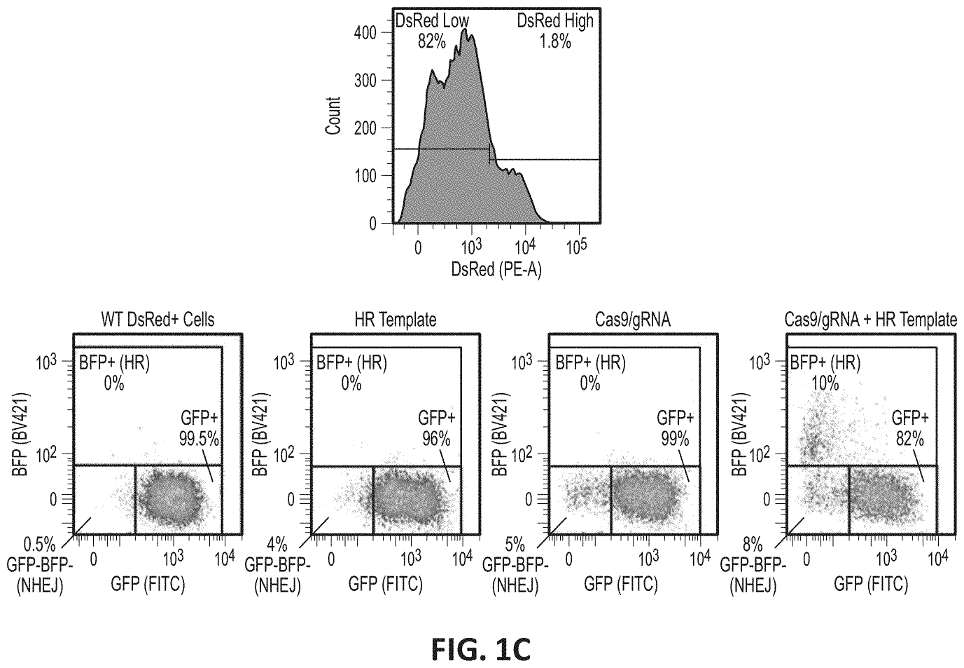

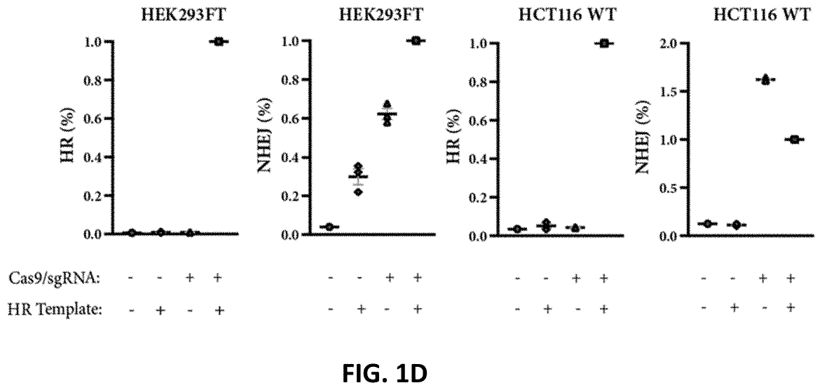

The RepairSwitch assay was developed to measure the balance of HR- and NHEJ-mediated double-strand break (DSB) repair. To accomplish this, it was necessary to design assay vectors that could be lenti-virally transduced into cells and would thereby be incorporated into the genome. It was determined that a fluorescent dual reporter system, using CMV-DsRed and EF1a-EGFP, would be most effective (FIG. 1A). This design gave a strong expression of both fluorescent markers. Additionally, it allowed DsRed to serve as a transduction control to ensure vector integration at an MOI of 0.3, while EGFP served as a target for CRISPR/Cas9 induced DSB. Utilizing the CRISPR/Cas9 system as a means of DNA damage induction allowed to ensure targeted DSB events to the fluorophore of the EGFP locus at a rate of 1 DSB per cell. Additionally, in conjunction with the administration of the CRISPR/Cas9 EGFP gRNA, exogenous single stranded DNA oligos were administered in excess and act as the repair template. In this system, the resulting fluorescence, post CRISPR/Cas9 cutting at the target locus, is dependent on the method of repair. Upon utilization of the repair template in repair via homologous recombination (HR) the resulting locus will be converted from EGFP to BFP. This is possible due to the sequence similarity of these fluorescent proteins, which differ in only two bases, resulting in the substitution of two adjacent amino acids. However, if the repair template is not utilized and repair occurs via the non-homologous end-joining pathway (NHEJ), there are two possible outcomes. Either, the EGFP fluorescence will be extinguished due to the error prone repair, or it is possible for the EGFP protein to be repaired correctly or with a silent mutation via NHEJ, thereby producing a viable fluorescent signal. However, those rare events will be indistinguishable from the DsRed+GFP+ population comprised of cells that did not experience cutting by CRISPR/Cas9. This can be due to lack of transfection by the vector or due to inaccessibility of the EGFP locus resulting from its integration in a heterochromatic region of the genome. A schematic of the RepairSwitch assay readout (FIG. 1B) provides a concise overview of the possible fluorescent outcomes and their respective implications.

### Example 2—Assay Performance with Control/Reference Samples

This assay was performed in both HEK293FT and HCT116 cells. Flow cytometry was used to quantify results, utilizing a gating strategy designed to reduce noise while maintaining a high degree of sensitivity, detecting rare events in the 1 in 10,000 range (FIG. 1C). The results show that the expression of BFP, indicating HR has occurred, will only take place in the presence of both the CRISPR/Cas9 EGFP gRNA and the repair template (FIG. 1D). However, this is not the case for NHEJ. In HEK293FT but not HCT116 cells, EGFP signal loss is increased upon transfection of the ssDNA oligo repair template alone, indicating NHEJ has occurred at the EGFP locus without the presence of the CRISPR/Cas9 EGFP gRNA. As expected, in both cell types, the CRISPR/Cas9 EGFP gRNA alone induces loss of EGFP signal as a result of repair by NHEJ, as no repair template is available. Interestingly, the addition of both the repair template and the CRISPR/Cas9 gRNA has an additive effect on the rate NHEJ in HEK293FT cells, as seen by the higher rates of NHEJ when in combination with either of the components on their own. However, this was not observed in HCT116 cells. In HCT116 cells, the addition of both repair template and CRISPR/Cas9 gRNA results in a lower rate of NHEJ than with the gRNA alone. These results show that the RepairSwitch assay is both modular and sensitive.

### Example 3—Impact of Genetic Modulation of Repair Factors on Rates of Repair

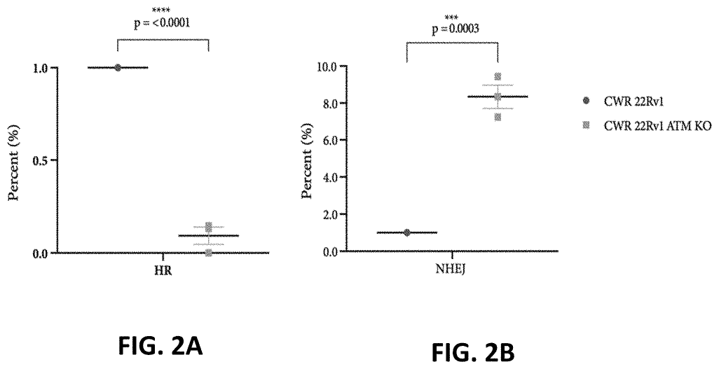

In order to verify the RepairSwitch assay's efficacy in measuring rates of HR and NHEJ, the impact of genetic modulation of ATM was tested, an important and well-studied HR repair protein, in CWR22Rv1 cell lines. As expected, there was a significant reduction in the rate of HR in the ATM−/− line as compared to WT (FIG. 2A). However, surprisingly, there was also a significant increase in the rate of NHEJ in the ATM−/− cells as compared to WT (FIG. 2B).

### Example 4—Impact of Pharmacological Inhibition of DNA Repair Enzymes on Rates of Repair

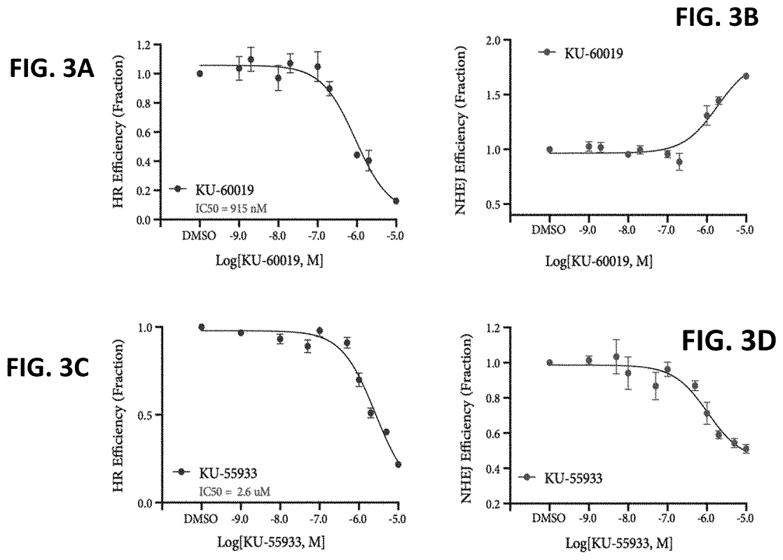

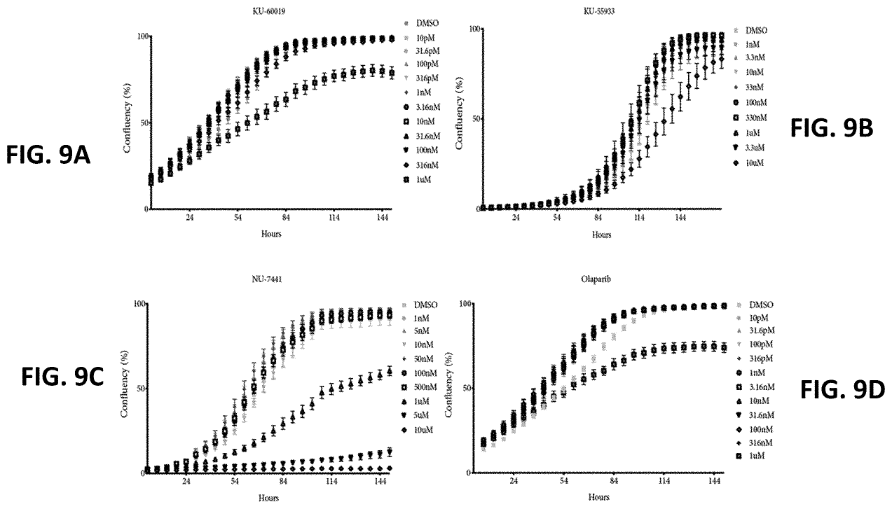

Utilizing a number of drugs targeting several repair factors as detailed in Table 1, the impact of pharmacological inhibition on rates of HR and NHEJ in HEK293FT cells was tested. Continuing the focus on ATM, two drugs that target the ATM protein were used, with varying degrees of potency and specificity, to ascertain whether pharmacological inhibition produced similar results to genetic manipulation of the ATM protein. KU-60019 is a potent and specific ATM inhibitor, while KU-55933 is both less potent and less specific. To ensure these drugs were non-toxic to the cells, proliferation was assayed using the Incucyte system, which showed that these drugs minimally attenuate cell growth even at higher doses. These results can be found in the supplemental material (FIGS. 9A-9B). For both KU-60019 and KU-55933, HR was reduced in a dose responsive manner with an IC50 of 915 nM and 2.6 uM respectively (FIG. 3A-3C). However, rates of NHEJ for the two drugs differed due to their difference in target specificity. The high degree of specificity of KU-60019 for ATM and the HR pathway, can be observed by the lack of negative impact on the rate of NHEJ with its usage. Interestingly, the rate of NHEJ can be seen to increase in a dose responsive manner as a result of KU-60019 usage. These results are consistent with our previous findings regarding the impact of genetic manipulation of ATM on the rates of HR and NHEJ. In contrast, KU-55933 has been shown to have off target effects on DNA-PK at high doses, an important component of the NHEJ pathway. This is reflected in the dose responsive reduction in rates of NHEJ in following treatment with this drug (FIG. 3D).

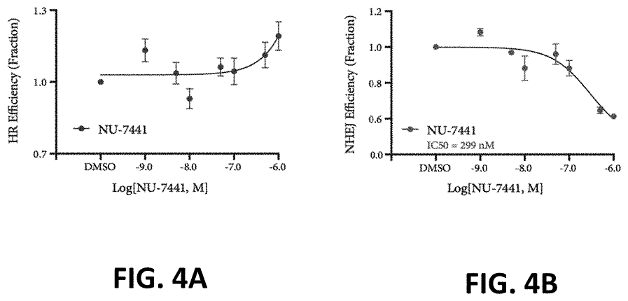

The pharmacological inhibition of NHEJ components was then observed, namely DNA-PK, to assess its impact on rates of HR and NHEJ in HEK293FT cells. To do so, NU-7441 was utlized, a potent and specific inhibitor of DNA-PK. Incucyte cell proliferation assays were performed to assess the toxicity of this drug, and it was found to inhibit cell growth at higher doses (FIG. 9C). Therefore, doses that were high enough to impact cell growth were removed from this dose curve so as not to impact the data. As expected, the usage of this DNA-PK inhibitor results in a dose-dependent reduction in rates of NHEJ (FIG. 4A). However, interestingly, rates of HR also increase in a dose responsive manner. This is similar to the effect on NHEJ seen when components of HR are inhibited.

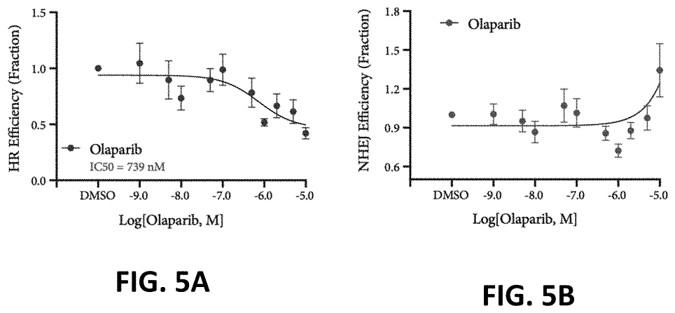

Lastly, the RepairSwitch assay was used to provide insight into the repair function of PARP by evaluating the impact of its inhibition on rates of HR and NHEJ. These results show a subtle reduction in rates of HR and a corresponding subtle increase in the rates of NHEJ (FIG. 5A-5B). Proliferation was assessed during Olaparib treatment as well, and shows it to minimally attenuate cell growth at higher doses (FIG. 9D).

### Example 5—Impact of Donor Template DNA Methylation on Rates of Repair

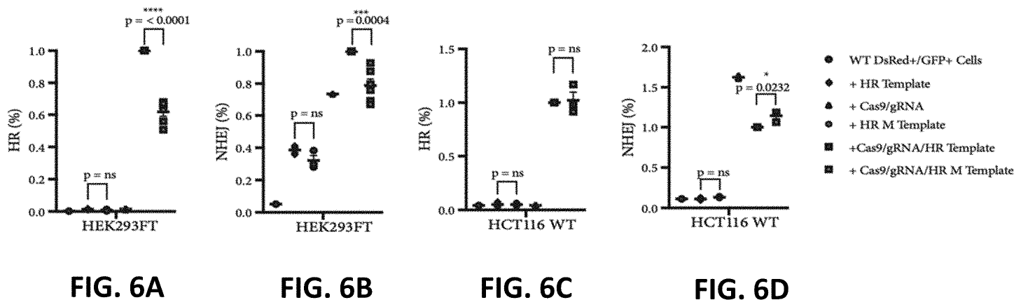

As shown previously, the RepairSwitch Assay is designed to be versatile, and can be used to explore numerous variables that may impact rates of repair by HR and NHEJ. Here the impact of donor template DNA methylation on rates of repair was investigated. This was accomplished by using donor template DNA that is either devoid of methylation or fully methylated at all seven CpG sites within the approximately 100 bp ssOligo. This experiment was performed in both HEK293FT cells (FIG. 6A-6B) and HCT116 cells (FIG. 6C-6D). These results show a significant decrease in the rates of both HR (p=<0.0001) and NHEJ (p=0.0004) in HEK293FT cells when a methylated template is provided versus an unmethylated one. However, the same cannot be said of HCT116 cells, in which there was no significant difference in HR with the methylated template as compared to unmethylated. Additionally, NHEJ (p=0.0232) repair shows a small but significant increase in rates when using the methylated template as compared to unmethylated. To ensure that these results are not due to differences in how the cells recognize methylated versus unmethylated donor template DNA, statistical analysis was performed on samples that received the control template alone (FIG. 6A-6D). These results showed that there were no significant differences in the rates of either HR or NHEJ upon use of either template.

### Example 6—Impact of DNMT Inhibitors on Rates of Repair

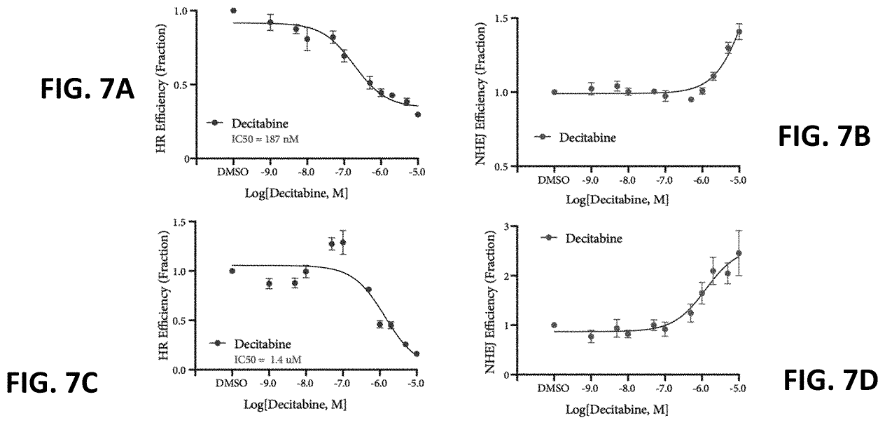

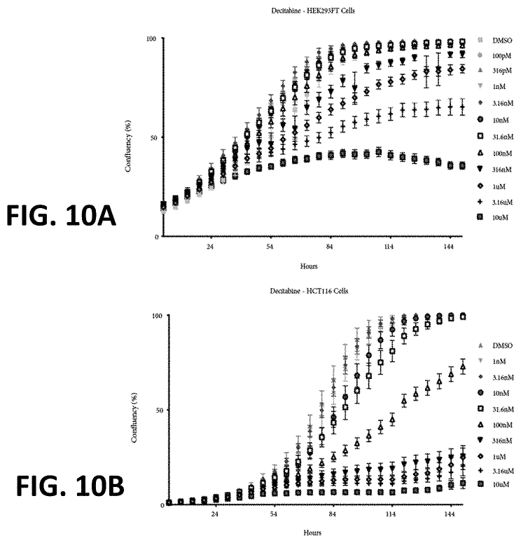

In order to investigate the potential role of the DNA methylation machinery in repair, pharmacological inhibition of DNA-methyltransferases was used to assess the impact of loss of these pathway components on rates of HR and NHEJ. This experiment was performed in both HEK293FT and HCT116 cells. As DNA-methyltransferase inhibitors can be toxic to cells, Incucyte cell proliferation assays were performed to ensure doses at which cell growth was inhibited were excluded from our analysis (FIG. 10A-10B). Decitabine, a cytidine analog that traps DNMTs on the DNA, was used in these experiments. Tesults show that treatment with Decitabine results in a dose responsive decrease in the rates of HR, and a compensatory increase in the rates of NHEJ in HEK239FT cells (FIG. 7A-7B). These results were also consistent in HCT116 cells (FIG. 7C-7D).

### Example 7—Impact of Genetic Manipulation of DNMT Genes on Rates of Repair

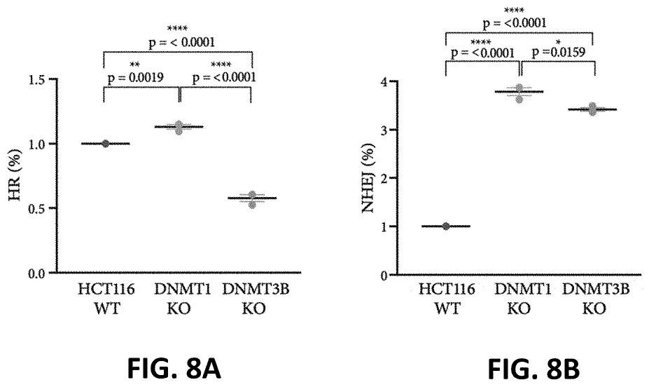

In order to get a clearer understanding of the role of DNMTs in repair, it was determined whether these results are consistent when genetic manipulation of these components are applied. Pharmacological inhibition and genetic manipulation are complementary approaches with distinct differences. Where pharmacological inhibition of DNMTs will trap these proteins and deplete them through proteolytic degradation, genetic manipulation will remove the proteins entirely and prevent any engagement with DNA to begin with. Using HCT116 WT, DNMT1 KO, and DNMT3B KO cells, rates of HR and NHEJ (FIG. 8A-8B) were compared. DNMT1 KO cells had slightly increased rates of HR as compared to WT, whereas DNMT3B KO cells exhibited a drastic decrease in rates of HR as compared to WT HCT116 cells. NHEJ was significantly increased in both HCT116 DNMT KO and DNMT3B KO cells as compared to WT. This is consistent with what was seen with pharmacological inhibition using Decitabine.

What is claimed is:

1. A method for determining a rate of DNA double strand repair on a DNA strand in a cell, the method comprising:
(a) delivering a reporter gene, a gene-editing agent, and a gene-repair template into a cell, wherein the gene-editing agent generates a DNA double strand break on the DNA strand;
(b) detecting a change in reporter gene expression, wherein the change in reporter gene expression indicates the presence of a DNA double strand repair event; and
(c) analyzing the change in reporter gene expression, thereby determining the rate of DNA double strand repair on the DNA strand in the cell.

2. The method of claim 1, wherein the reporter gene comprises a DsRed, an EGFP, a BFP reporter gene, or any combinations thereof.

3. The method of claim 1, wherein the gene-editing agent comprises CRISPR/Cas9 components.

4. The method of claim 3, wherein the gene-editing agent further comprises a guide RNA (gRNA), wherein the gRNA is targeted to an individual gene of the cell.

5. The method of claim 4, wherein the gRNA is targeted to the EGFP reporter gene.

6. The method of claim 1, wherein the gene-repair template comprises an exogenous single stranded DNA oligonucleotide.

7. The method of claim 6, wherein the exogenous single stranded DNA oligonucleotide is about 100 base pairs in length.

8. The method of claim 1, wherein the delivering comprises a virus-based delivery.

9. The method of claim 8, wherein the virus-based delivery utilizes a lentivirus.

10. The method of claim 1, wherein the change in reporter gene expression comprises a change in fluorescence from the reporter gene.

11. The method of claim 10, wherein the change in fluorescence comprises change from a green fluorescent protein to a blue fluorescent protein.

12. The method of claim 10, wherein the change in fluorescence comprises loss of a green fluorescent protein.

13. The method of claim 1, wherein the DNA double strand repair event comprises homologous recombination (HR) or non-homologous end joining (NHEJ).

14. The method of claim 1, wherein the analyzing comprises flow cytometry analysis.

15. The method of claim 1, wherein the cell is from a HEK293FT or a HCT116 cell line.

16. The method of claim 1, wherein the DNA strand comprises a genetic alteration.

17. The method of claim 1, wherein the DNA strand comprises an epigenetic alteration.

18. The method of claim 17, wherein the epigenetic alteration comprises DNA methylation.

19. The method of claim 1, wherein the DNA strand comprises a pharmacologic alteration.

20. The method of claim 19, wherein the pharmacologic alteration comprises inhibition of DNA repair enzymes in the cell.
